(19) United States
(12) Patent Application Publication
(10) Pub. No.: US 2016/0158471 A1
(43) Pub. Date: Jun. 9, 2016

(54) INTEGRATION OF GLUCOSE DATA TO ADJUST INHALED INSULIN DOSE

(71) Applicant: Dance Biopharm, Inc., Brisbane, CA (US)

(72) Inventors: Jim Fink, San Mateo, CA (US); Michael Shawver, Mill Valley, CA (US); Lisa Molloy, San Mateo, CA (US); Ben Stedman, Palo Alto, CA (US)

(73) Assignee: Dance Biopharm, Inc., Brisbane, CA (US)

(21) Appl. No.: 14/958,318

(22) Filed: Dec. 3, 2015

Related U.S. Application Data
(60) Provisional application No. 62/088,260, filed on Dec. 5, 2014.

(51) Int. Cl. A61M 15/00; A61K 9/00; A61M 11/00; A61K 38/28

(52) U.S. Cl. CPC A61M 15/0066; A61K 38/28; A61K 9/0078; A61M 11/005; A61M 15/0021; A61M 15/0091; A61M 2202/00; A61M 2230/201; A61M 2230/005; A61M 2205/3584; A61M 2230/40; A61M 2205/502; A61M 2205/52

(57) ABSTRACT

An insulin dispenser receives blood glucose readings wirelessly from a blood glucose monitor, and calculates an insulin dose based on the historical response of the particular patient to prior insulin doses. The insulin dispenser preferably nebulizes an insulin-containing fluid for inhalation by the patient.

This application claims the benefit of U.S. Provisional Patent Application No. 62/088,260, filed Dec. 5, 2014, and titled “Integration of Glucose Data to Adjust Inhaled Insulin Dose,” the entire disclosure of which is hereby incorporated by reference herein for all purposes.

### BACKGROUND OF THE INVENTION

There is a global epidemic of diabetes. The estimated 382 million people with diabetes worldwide is expected to grow to 592 million in less than 25 years. Diabetes patients account for up to one third of healthcare costs in many regions the world over. A diagnosis of type 2 diabetes after the age of 40 leads to an average decrease in lifespan of 13-14 years, as well as deterioration in quality of life. But numerous studies have shown that better glucose control can extend life expectancy, improve quality of life, and reduce the costs of managing diabetes and its complications.

The long-term benefits of mealtime insulin therapy to control blood glucose for type 2 patients have been consistently demonstrated. Many clinical studies have shown that insulin is not only most effective in controlling blood glucose, but also preserves pancreatic beta-cell function (insulin production), and slows progression. Although injected insulin is the gold standard for treatment, traditionally it has been the last drug taken by Type 2 patients (who make up about 90-95% of diabetes). Typically, patients delay taking mealtime insulin for 5-10 years, in order to avoid multiple daily injections as long as possible. Delaying insulin treatment or refusing to take injections eventually results in significant health consequences for the patients and enormous costs to health care systems.

Now all of the major diabetes medical associations recommend the introduction of insulin much earlier in the treatment process for type 2 patients, and if glycosylated hemoglobin (HbA1c) is above 9% upon diagnosis and patients are symptomatic, insulin is recommended immediately.

In addition, around 3 million people living with type 1 diabetes in America depend on taking insulin daily via injections or infusion pumps to keep them alive. This daily process is cumbersome, uncomfortable, and expensive.

### BRIEF SUMMARY OF THE INVENTION

According to one aspect, an insulin dispenser comprises a reservoir for holding an insulin-containing liquid, and a nebulizer for generating a mist of the insulin-containing liquid. The insulin dispenser further includes a wireless communication transceiver, a microprocessor, and a memory. The memory holds instructions that, when executed by the microprocessor cause the insulin dispenser to, under control of the microprocessor, receive data from a blood glucose monitor via the wireless communication transceiver. The data indicate measurements of the blood glucose level of a patient over time, including during at least one time period after the patient has received a dose of insulin from the dispenser. The instructions further cause the insulin dispenser to record information describing one or more prior insulin doses dispensed to the patient by the insulin dispenser, and characterize, using the data received from the blood glucose monitor, the patient's response to the one or more prior insulin doses. The instructions further cause the insulin dispenser to receive a current reading of the patient's blood glucose level from the blood glucose monitor via the wireless communication transceiver, compute a new insulin dose based in part on the characterization of the patient's response to the one or more prior insulin doses and based in part on the current reading of the patient's blood glucose level, and dispense the dose of insulin so that an amount of insulin-containing liquid is nebulized corresponding to the computed new insulin dose, for inhalation by the patient. In some embodiments, the nebulizer further comprises a vibratable mesh plate and a dispensing mechanism for dispensing the insulin-containing liquid from the reservoir to the vibratable plate, such that the insulin-containing liquid is nebulized when the vibratable mesh plate is vibrated. The wireless communication transceiver may be a Bluetooth communication transceiver. In some embodiments, the insulin dispenser is combined with the blood glucose monitor. In some embodiments, the blood glucose monitor performs continuous blood glucose monitoring. In some embodiments, the insulin dispenser further comprises a mouthpiece through which the nebulized insulin-containing liquid is supplied to the patient and a flow sensor that detects inspiratory flow of the patient inhaling through the mouthpiece, and the nebulizer nebulizes the insulin-containing liquid only during a time or times when the flow sensor indicates that the patient is inhaling through the mouthpiece. In some embodiments, the nebulization continues during a time or times when the flow sensor indicates that the patient is inhaling through the mouthpiece, until the dose has been nebulized. In some embodiments, the insulin dispenser further comprises a display and one or more user controls, and the instructions, when executed by the microprocessor, further cause the insulin dispenser to display the computed new insulin dose on the display and to receive, via the one or more user controls, an adjustment to the computed dose to specify an adjusted dose before dispensing adjusted dose of insulin-containing liquid.

According to another aspect, a method of controlling glucose levels in a patient comprises receiving, via a wireless communication transceiver, data from a blood glucose monitor. The data indicate the blood glucose level of a patient over time, including during at least one time period after the patient has received a dose of insulin from the dispenser. The method further comprises recording information describing one or more prior insulin doses dispensed to the patient by the insulin dispenser and characterizing, using the data received from the blood glucose monitor, the patient's response to the one or more prior insulin doses. The method further comprises receiving a current reading of the patient's blood glucose level from the blood glucose monitor via the wireless communication transceiver, computing a new insulin dose based in part on the characterization of the patient's response to the one or more prior insulin doses and based in part on the current reading of the patient's blood glucose level, and dispensing the new insulin dose to the patient. In some embodiments, dispensing the new insulin dose to the patient comprises supplying an insulin-containing liquid to one side of a vibratable mesh plate, and vibrating the vibratable mesh plate during successive inhalations of the patient until the complete dose has been nebulized. In some embodiments, the insulin-containing liquid is supplied to the vibratable mesh plate at a rate that is at or below the rate at which the insulin-containing liquid is nebulized by the vibrating mesh plate. In some embodiments, liquid is supplied to the vibratable mesh plate only during patient inhalation and the vibratable mesh plate is vibrated only during patient inhalation. In some embodiments, the method further comprises displaying the computed dose on a display. In some embodiments, the method further comprises receiving, via user controls, and adjustment of the computed dose.

According to another aspect, a system comprises a glucose monitor and an insulin dispenser. The insulin dispenser comprises a reservoir for holding an insulin-containing liquid, a vibratable mesh plate, and a dispensing mechanism for dispensing the insulin-containing liquid from the reservoir to the vibratable plate, such that a the insulin-containing liquid is nebulized when the vibratable mesh plate is vibrated. The insulin dispenser further comprises a wireless communication transceiver, a microprocessor, and a memory. The memory holds instructions that, when executed by the microprocessor cause the insulin dispenser to, under control of the microprocessor, receive data from a blood glucose monitor via the wireless communication transceiver the data indicating measurements of the blood glucose level of a patient over time, including during at least one time period after the patient has received a dose of insulin from the dispenser. The instructions further cause the insulin dispenser to record information describing one or more prior insulin doses dispensed to the patient by the insulin dispenser, and characterize, using the data received from the blood glucose monitor, the patient's response to the one or more prior insulin doses. The instructions further cause the insulin dispenser to receive a current reading of the patient's blood glucose level from the blood glucose monitor via the wireless communication transceiver, compute a new insulin dose based in part on the characterization of the patient's response to the one or more prior insulin doses and based in part on the current reading of the patient's blood glucose level, and dispense the dose of insulin to the vibratable mesh plate so that an amount of insulin-containing liquid is nebulized corresponding to the computed new insulin dose, for inhalation by the patient.

### BRIEF DESCRIPTION OF THE DRAWINGS

FIG. 1 illustrates a system in which the invention may be embodied.

FIG. 2 illustrates an example user interface, according to an embodiment of the invention.

FIG. 3 illustrates the dispensing of insulating-containing liquid in accordance with embodiments of the invention.

### DETAILED DESCRIPTION OF THE INVENTION

For many diabetes patients, management of their disease is a cumbersome, time-consuming, inconvenient, and error-prone process. Frequent blood sugar measurements are necessary, often involving drawing a small amount of blood. The patient may then be required to interpret the blood sugar data, and compute a dose of insulin intended to control the patient's blood sugar to a target level. Several doses of insulin may be required per day.

Many different insulin delivery methods are available, depending on the severity of the patient's condition and the patient's preference. Many patients give themselves insulin injections regularly, using hypodermic syringes. Some patients may use an insulin pen that automates the injection of insulin through the skin. Other dispensing devices inject a high-pressure insulin-containing mist into the skin without the use of a needle. Patients with severe conditions, for example patients with Type I diabetes whose bodies can no longer produce insulin at all, may wear an insulin pump that continually supplies insulin to the body.

One particularly convenient insulin delivery method is via inhalation. An insulin-containing liquid is nebulized and the resulting mist is inhaled by the patient. Inhaled insulin may be especially fast acting as compared with insulin delivered by other methods, and the inhalation delivery technique is painless and convenient.

Blood glucose monitors have recently become available that can report blood glucose readings to other devices via wireless electronic communications, for example Bluetooth® technology as specified by the Bluetooth SIG, Inc., of Kirkland, Wash., USA. Bluetooth communications typically have a range of up to several meters.

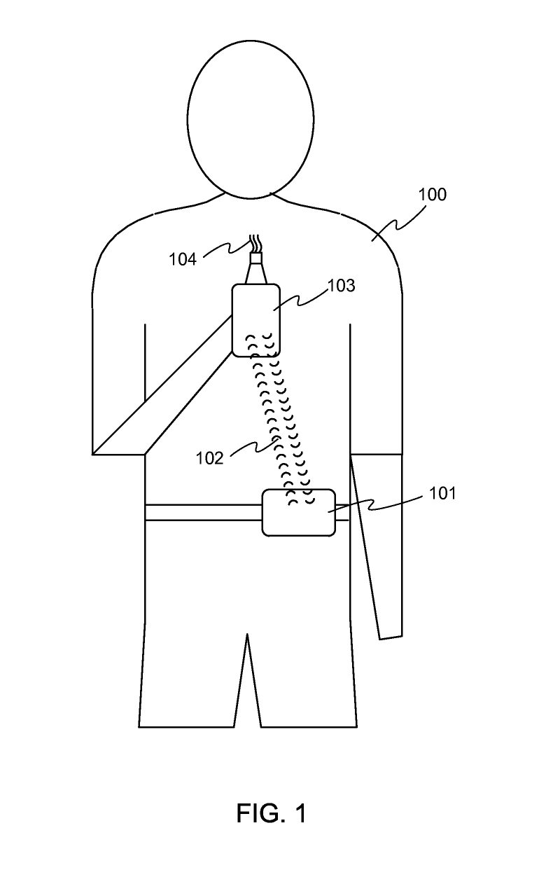

FIG. 1 illustrates a system in which the invention may be embodied.

In the example of FIG. 1, a patient 100 uses a blood glucose monitor 101. As illustrated, blood glucose monitor 101 may be a continuous monitor, having a probe embedded in the skin of patient 100 and taking readings of the patient's blood glucose level every few minutes. Blood glucose monitor 101 also includes a wireless communication transceiver, enabling blood glucose monitor 101 to transmit readings over a wireless communication link 102, which may be, for example, a Bluetooth link of another kind of wireless communication link. In other embodiments, a non-continuous blood glucose monitor could be used.

Whichever kind of blood glucose monitor is used, readings of the patient's blood glucose level are periodically taken and transmitted to an insulin dispenser 103. Insulin dispenser 103 is preferably an inhalation-type dispenser. This kind of dispenser stores a quantity of insulin-containing liquid in a reservoir, and nebulizes the insulin-containing liquid for inhalation by the patient. In a nebulizer according to some embodiments, a dispensing device provides the liquid to one side of a vibratable mesh plate. Preferably, the liquid is provided to the vibratable plate as a volumetric dose. To dispense insulin, the vibratable mesh plate is vibrated such that the insulin-containing liquid is drawn through the openings in the mesh plate and nebulized from the other side of the plate. The resulting mist 104 can simply be inhaled by the patient. Typically, the plate is vibrated at ultrasonic frequencies. Some ultrasonic dispensing devices suitable for dispensing insulin via inhalation are described in U.S. Pat. No. 7,360,536 to Patel et al., issued Apr. 22, 2008, and U.S. Pat. No. 8,555,874 to Fink et al., issued Oct. 15, 2013, the entire disclosures of which are hereby incorporated by reference herein. Other suitable kinds of nebulizers may be used as well. Insulin dispenser 103 can preferably administer a wide range of doses, for example from 1 to 40 or more IU equivalents. The dosage may be adjusted, for example, by controlling the amount of time the vibratable plate is vibrated for a particular dose.

The blood glucose data received by insulin dispenser 103 preferably includes one or more readings taken in a time period after the administration of a dose of insulin to the patient by insulin dispenser 103.

Different patients react differently to insulin, such that a particular insulin dose may be more effective for one patient than for another. This between-patient variability is substantial. For this reason, simply basing an insulin dose on the patient's current blood glucose level may not result in optimum dosing for any one particular patient. However, evidence suggests that the variability of a particular patient's response to insulin is small in comparison to the between-patient variability. Embodiments of the invention therefore monitor a patient's blood glucose levels over time, including after insulin dosing, and characterize the patient's response to insulin. When another dose is required, the dosage is calculated based not only on the patient's current blood glucose level, but also on the particular patient's prior responses to insulin doses.

In one simple algorithm, insulin dispenser 103 may record the timing and quantity of each insulin dose, and also record the timing and values of the blood glucose readings received from blood glucose monitor 101. The effectiveness of each dose may be measured by comparing the most recent blood glucose reading before a particular dose with a reading taken a predetermined time after the particular dose, for example one hour after the dose. This change in blood glucose level may be plotted or otherwise correlated against the dosage to characterize the particular patient's response to insulin. When subsequent blood glucose readings indicate that another dose of insulin is needed, the plot or correlation can be consulted to determine the dose that has historically resulted in the desired reduction of blood glucose level. The calculated dose can then be dispensed to the patient. The pertinent information about this dose is also recorded, and becomes historical data to be used in the calculation of future doses. Thus, the characterization of the patient's response to insulin may be continually refined over time, as the patient uses insulin dispenser 103.

It will be understood that other algorithms may be used in embodiments of the invention, and that algorithms may incorporate other kinds of parameters in determining an appropriate dose. Other parameters that may be considered could include, for example, one or more parameters selected from the patient's weight, the time of day, the time since the patient last ate a meal, the patient's activity level, or other parameters.

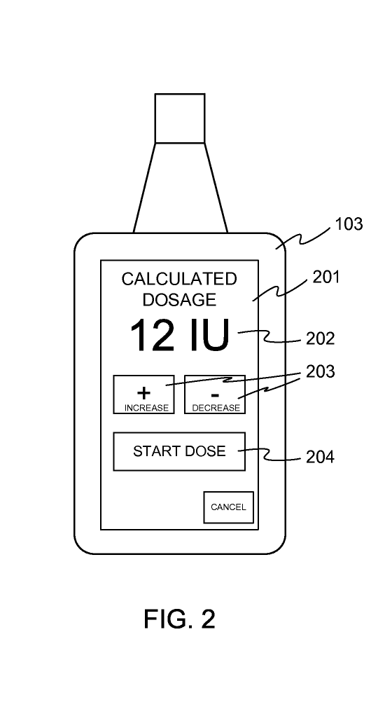

Preferably, patient 100 is offered a chance to review the calculated dosage before it is administered. FIG. 2 illustrates an example user interface that may be presented to patient 100 for this purpose. For example, insulin dispenser 103 may include a touchscreen display 201, on which the calculated dose 202 is prominently displayed. Patient 100 may be given the opportunity to adjust the dosage (for example based on his or her experience with diabetes management) using menu selections such as buttons 203, and can then signal that the dose should start 204 when patient 100 is prepared for inhalation. It will be understood that the user interface shown in FIG. 2 is merely an example, and that a wide variety of other user interfaces may be provided in embodiments of the invention. For example, insulin dispenser 103 may include electromechanical buttons usable by the patient to enter information, rather than using touchscreen selections. Similarly, additional or different information may be shown on touchscreen 202, and other displays and menus may be presented for configuring the device, reviewing data, or for other purposes. Many other variations are possible.

In some embodiments, the data stored in insulin dispenser 103, including the historical blood glucose readings and provided insulation dosages and timing, may be read out of insulin dispenser 103 for external storage and analysis. For example, the patient's physician may wish to review the stability of the patient's blood glucose levels, to determine if adjustments to treatment may be appropriate.

Thus, administration of insulation to the patient may be feedback controlled, and tailored to the patient's particular physiology.

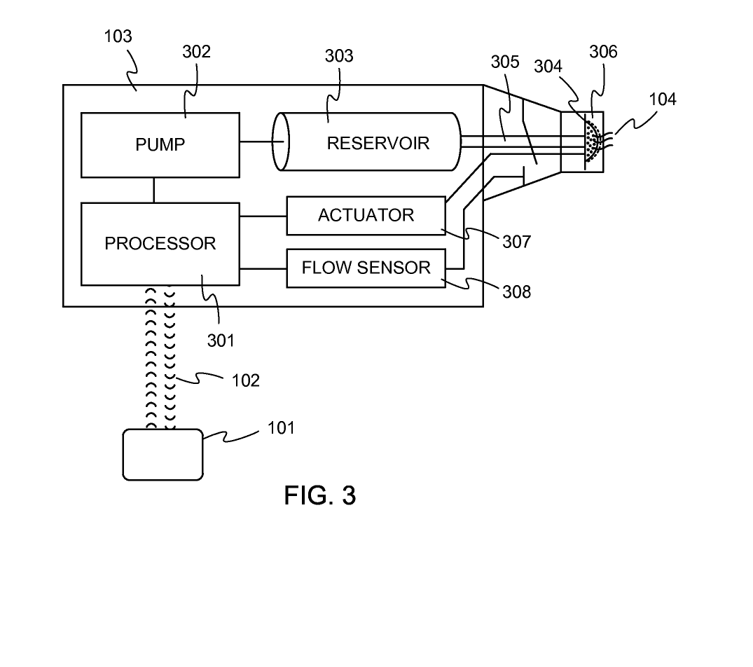

FIG. 3 illustrates the dispensing of insulin-containing liquid in more detail, in accordance with embodiments of the invention. Insulin dispenser 103 receives one or more blood glucose readings from glucose monitor 101 via wireless link 102. Insulin dispenser 103 includes a processor 301, for example a microprocessor or microcontroller and supporting circuitry such as memory (not shown). Processor 301 calculates a proper insulin dose as described above, for example based on the patient's current blood glucose level and reactions to previous insulin doses.

Processor 301 controls a pump 302 to dispense the calculated dose of liquid from reservoir 303 to vibratable mesh plate 304 through path 305. Vibratable mesh plate 304 is preferably in or near a mouthpiece 306, through which the patient inhales. Pump 302 may be any suitable kind of pump, for example a screw-type or gear-type positive displacement pump or another kind of pump that can dispense calculated amounts of liquid from reservoir 303.

An actuator 307 can cause vibratable mesh plate 304 to vibrate, under control of processor 301. For example, actuator 307 may include a piezoelectric element (not shown) near vibratable mesh plate 304 and associated support electronics. A flow sensor 308 detects whether the patient is inhaling or not, and provides this information to processor 301. Preferably, liquid from reservoir 303 is dispensed and vibratable plate 304 is vibrated only during actual inhalation by the patient. Dispensing may occur during several successive inhalations until the proper dose is completely dispensed. Insulin dispenser 103 may signal the patient when the providing is complete, for example using an indicator light, a sound, or another indicator.

The invention has now been described in detail for the purposes of clarity and understanding. However, those skilled in the art will appreciate that certain changes and modifications may be practiced within the scope of the appended claims. It is to be understood that any workable combination of the features and capabilities disclosed above in the various embodiments is also considered to be disclosed.

What is claimed is:

1. An insulin dispenser, comprising:
a reservoir for holding an insulin-containing liquid;
a nebulizer for generating a mist of the insulin-containing liquid;
a wireless communication transceiver;
a microprocessor; and
a memory, the memory holding instructions that, when executed by the microprocessor cause the insulin dispenser to, under control of the microprocessor:
receive data from a blood glucose monitor via the wireless communication transceiver, the data indicating measurements of the blood glucose level of a patient over time, including during at least one time period after the patient has received a dose of insulin from the dispenser;
record information describing one or more prior insulin doses dispensed to the patient by the insulin dispenser;
characterize, using the data received from the blood glucose monitor, the patient's response to the one or more prior insulin doses;
receive a current reading of the patient's blood glucose level from the blood glucose monitor via the wireless communication transceiver;
compute a new insulin dose based in part on the characterization of the patient's response to the one or more prior insulin doses and based in part on the current reading of the patient's blood glucose level; and
dispense the dose of insulin so that an amount of insulin-containing liquid is nebulized corresponding to the computed new insulin dose, for inhalation by the patient.

2. The insulin dispenser of claim 1, wherein the nebulizer further comprises:
a vibratable mesh plate; and
a dispensing mechanism for dispensing the insulin-containing liquid from the reservoir to the vibratable plate, such that the insulin-containing liquid is nebulized when the vibratable mesh plate is vibrated.

3. The insulin dispenser of claim 1, wherein the wireless communication transceiver is a Bluetooth communication transceiver.

4. The insulin dispenser of claim 1, in combination with the blood glucose monitor.

5. The combination of claim 4, wherein the blood glucose monitor performs continuous blood glucose monitoring.

6. The insulin dispenser of claim 1, further comprising:
a mouthpiece through which the nebulized insulin-containing liquid is supplied to the patient; and
a flow sensor that detects inspiratory flow of the patient inhaling through the mouthpiece;
wherein the nebulizer nebulizes the insulin-containing liquid only during a time or times when the flow sensor indicates that the patient is inhaling through the mouthpiece.

7. The insulin dispenser of claim 6, wherein the nebulization continues during a time or times when the flow sensor indicates that the patient is inhaling through the mouthpiece, until the dose has been nebulized.

8. The insulin dispenser of claim 1, further comprising:
a display; and
one or more user controls;
wherein the instructions, when executed by the microprocessor further cause the insulin dispenser to display the computed new insulin dose on the display; and
receive, via the one or more user controls, an adjustment to the computed dose to specify an adjusted dose before dispensing adjusted dose of insulin-containing liquid.

9. A method of controlling glucose levels in a patient, the method comprising:
receiving, via a wireless communication transceiver, data from a blood glucose monitor, the data indicating the blood glucose level of a patient over time, including during at least one time period after the patient has received a dose of insulin from the dispenser;
recording information describing one or more prior insulin doses dispensed to the patient by the insulin dispenser;
characterizing, using the data received from the blood glucose monitor, the patient's response to the one or more prior insulin doses;
receiving a current reading of the patient's blood glucose level from the blood glucose monitor via the wireless communication transceiver;
computing a new insulin dose based in part on the characterization of the patient's response to the one or more prior insulin doses and based in part on the current reading of the patient's blood glucose level; and
dispensing the new insulin dose to the patient.

10. The method of claim 9, wherein dispensing the new insulin dose to the patient comprises:
supplying an insulin-containing liquid to one side of a vibratable mesh plate; and
vibrating the vibratable mesh plate during successive inhalations of the patient until the complete dose has been nebulized.

11. The method of claim 10, wherein the insulin-containing liquid is supplied to the vibratable mesh plate at a rate that is at or below the rate at which the insulin-containing liquid is nebulized by the vibrating mesh plate.

12. The method of claim 11, wherein liquid is supplied to the vibratable mesh plate only during patient inhalation and the vibratable mesh plate is vibrated only during patient inhalation.

13. The method of claim 9, further comprising displaying the computed dose on a display.

14. The method of claim 13, further comprising receiving, via user controls, and adjustment of the computed dose.

15. A system, comprising:
a glucose monitor; and
an insulin dispenser;
wherein the insulin dispenser comprises:
a reservoir for holding an insulin-containing liquid;
a vibratable mesh plate;
a dispensing mechanism for dispensing the insulin-containing liquid from the reservoir to the vibratable plate, such that a the insulin-containing liquid is nebulized when the vibratable mesh plate is vibrated;
a wireless communication transceiver;
a microprocessor; and
a memory, the memory holding instructions that, when executed by the microprocessor cause the insulin dispenser to, under control of the microprocessor:
receive data from a blood glucose monitor via the wireless communication transceiver, the data indicating measurements of the blood glucose level of a patient over time, including during at least one time period after the patient has received a dose of insulin from the dispenser;
record information describing one or more prior insulin doses dispensed to the patient by the insulin dispenser;
characterize, using the data received from the blood glucose monitor, the patient's response to the one or more prior insulin doses;
receive a current reading of the patient's blood glucose level from the blood glucose monitor via the wireless communication transceiver;
compute a new insulin dose based in part on the characterization of the patient's response to the one or more prior insulin doses and based in part on the current reading of the patient's blood glucose level; and
dispense the dose of insulin to the vibratable mesh plate so that an amount of insulin-containing liquid is nebulized corresponding to the computed new insulin dose, for inhalation by the patient.
